(19) United States
(12) Patent Application Publication
(10) Pub. No.: US 2007/0178465 A1
(43) Pub. Date: Aug. 2, 2007

(54) N,n-dimethylacrylamide-based high molecular weight polymer

(75) Inventors: Jan Sudor, Toulouse (FR); Valessa Barbier, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: COMMISSARIAT A L'ENERGIE ATOMIQUE, Paris (FR)

(21) Appl. No.: 10/592,795

(22) PCT Filed: Mar. 10, 2005
(86) PCT No.: PCT/FR05/00573

§ 371 Date: Jan. 18, 2007

(30) Foreign Application Priority Data
Mar. 15, 2004 (FR) 0402630

(51) Int. Cl. C12Q 1/68; G01N 33/53; C08G 63/91

(52) U.S. Cl. 435/6; 435/7.1; 525/54.1; 525/54.2

(57) ABSTRACT

The invention relates to water-soluble N,N-dialkylacrylamide-based high molecular weight random polymers and monomers laterally functionalised by molecules of interest and to the use thereof for preparing solid supports provided with at least one surface on which said polymers are adsorbed. Said invention also relates to the thus modified solid supports, to a method for the production thereof and to different uses thereof, in particular for immobilising molecules of interest or for carrying out chemical, biochemical or biological reactions.

The present invention relates to high molecular weight random polymers based on N,N-dialkylacrylamide and monomers laterally functionalized with molecules of interest, and to their use for the preparation of solid supports having at least one surface on which said polymers are adsorbed. The present invention also relates to the solid supports thus modified and to the process for the production thereof, and to their various uses, in particular for the immobilization of molecules of interest or for performing chemical, biochemical or biological reactions.

In recent years, there has been increasing interest in DNA, protein or peptide chips which have radically revolutionized the experimental approach in molecular biology. Typically, such chips are produced starting from a small silicon-based (for example of glass) or plastic solid support, on which thousands of biological or chemical molecules of interest are immobilized.

Supports bearing these immobilized molecules of interest can advantageously be utilized for the detection and the recognition of biological species, but other applications for these supports, such as for example on-support chemical synthesis or modification or on-support enhancement of biological reactions and the digestion of proteins, are also possible.

With regard to these various applications, it is essential to have available functionalized solid supports displaying a certain number of qualities.

These supports must in particular enable the reproducible immobilization of molecules of interest, insofar as a reproducible immobilization is a precondition for a result, for example a detection, which is itself reproducible.

On the other hand, it is important to have available supports which can be prepared easily and rapidly, without the use of costly and complicated chemistry.

Finally, it can be advantageous to have available supports with reversible functionalization, that is to say supports which it is possible to regenerate by refunctionalizing the surface on which molecules of interest have been immobilized (regenerable supports).

Many methods enabling the immobilization of biological or chemical molecules on surfaces have been described in the literature for the purpose of performing biochemical reactions or of capturing, detecting or testing different target molecules. In the majority of cases, and in particular as regards “disposable”, that is to say single-use, DNA or protein chips, the biological molecules are immobilized on the support by means of a covalent bond in such a manner that the molecules do not detach from the support during the use of the chips, in particular during the washing steps. In this case, this immobilization is generally performed in two distinct steps:
a first step of functionalization of the supports which consists in chemical modification of their surface by grafting of synthetic molecules (coupling agents) which will ensure the attachment of the biological molecules onto the support;
a second immobilization step consisting in establishing a covalent bond between the biological molecules and these functionalized supports.

In the particular case of DNA chips which are designed for the purpose of detecting different sequences and variations of sequences, and this at many sites and simultaneously, methods utilizing polyacrylamide-based supports are among the most interesting for immobilizing oligonucleotides (ON) on the surface of the supports. As claimed in the literature, few non-specific interactions are observed with such surfaces and the chips which result therefrom display high efficacy and density of readily modulable probes. However, in spite of these advantages, the immobilization of probes on polyacrylamide-based surfaces necessitates the activation of the gels and/or the probes by complicated chemistry. It is thus a procedure that is difficult to perform. Another approach consists in the immobilization of the ON via copolymerization of ON functionalized with acrylamide with a crosslinked polyacrylamide (Rehman F. N., et al., Nucleic Acid Research, 1999, 27(2), 649-655). In this case, the gels can be prepared by standard polymerization techniques well known to the person skilled in the art such as radical polymerization. The surfaces thus prepared display a high density of stably attached probes. On the other hand, the surfaces of these supports have the disadvantage of being permanently modified and for this reason cannot be easily regenerated.

It has also already been proposed, in particular by Southern E. et al., Nat. Genet., 1999, 21, 5-9, that DNA chips be prepared by modifying the surface of a solid support with poly-L-lysine onto which ON can then be attached electrostatically. In this case, the surfaces can be regenerated quite easily, but the ON are in direct contact with the surface, which has adverse consequences on their capacity to hybridize with target complementary sequences in solutions on account of geometric constraints.

It is also possible to immobilize biotinylated ON or peptides on surfaces functionalized with streptavidin by making use of biotin-streptavidin coupling, or else on gold surfaces when the ON have previously been functionalized with a thiol group at one of their ends. The surfaces thus obtained using these techniques are also difficult to regenerate and require previous functionalization of the molecules before their attachment to the support.

Still in the same regard, novel synthetic polymers chemically conjugated with different proteins in order to create novel hybrid protein-polymer functional molecules have also been proposed. In this context, polyethylene glycol (PEG) conjugated with therapeutic proteins or enzymes such as for example catalase are among the most studied hybrid molecules (Abuchowski A. et al., J. Biol. Chem., 1977, 252, 3582-3586). While such polymer-protein conjugates generally display better stability, and greater solubility in organic solvents and in vivo, they nonetheless have the disadvantage of not being attached to the surface of a solid support and cannot therefore be utilized for the preparation of protein chips. Further, it has also already been proposed that this problem be remedied by proposing microfluidic systems of the “Labs-on-chips” type, in which enzymes are covalently immobilized on the surface of microcapillaries (see in particular Mao H et al., Anal Chem., 2002, 74, 379-385). Just like the DNA chips described above, these systems also have the disadvantage of being difficult to regenerate on account of the mode of attachment of the proteins by means of a covalent bond.

As claimed in yet another approach, different types of solid support whose surface is functionalized with homopolymers or copolymers having a plurality of ligand molecules which are capable of capturing target molecules in solutions have also already been proposed. Thus for example U.S. Pat. No. 5,695,936 describes a method for detection of a nucleotide sequence of interest with the aid of a nucleotide probe labeled with a tracer. As claimed in this method, a reagent consisting of a copolymer based on N-vinylpyrrolidone or maleic anhydride, having a molecular weight lying between 5000 and 400 000 g/mole and provided with lateral substituents of an oligonucleotide type, is used. The immobilization of this copolymer on the surface of the support is effected in a quite complicated manner by means of a complex between the solid support, a probe labeled with a tracer, a reagent and a target nucleic acid. The supports thus obtained display good detection sensitivity but their preparation is long and expensive and they are difficult to regenerate.

Moreover, U.S. Pat. No. 5,453,461 describes biologically active polymers of formula P-(A)_{q} wherein P represents a linear, branched or crosslinked polymer formed for example from acrylic monomers, A represents a biologically active section and can for example be biotin or an ON comprising from 1 to 80 nucleotide units and q is a whole number equal to 1 or 2. As claimed in the structure of these polymers, A is always located at the end of the polymer chain and not laterally. These polymers generally have a molecular weight lower than 1 000 000 and are intended to be attached covalently (by means of an amide, urea, ester or ether bond or a urethane group) to the surface of solid supports for the purpose of being used in biochemical identification reactions or in reactions with other biologically active molecules such as proteins or nucleic acids. Once again, this type of support is difficult to regenerate.

Finally, as claimed in a last approach, it has already been proposed to prepare solid supports whose surface is covered by a layer of polymers having biologically active substituents by simple adsorption of said polymers onto the surface of the support.

Thus, U.S. Pat. No. 5,723,344 describes the preparation of copolymers formed from an N-vinylpyrrolidone monomer and a second monomer containing a reactive functional group enabling the attachment by covalent bonding of a biological ligand capable of forming a complex with a target molecule such as for example an antigen/antibody, polynucleotide/polynucleotide, poly-nucleotide/nucleic acid, antibody/hapten or hormone/receptor complex, and their capacity to be adsorbed on the surface of solid supports. Such copolymers have a molecular weight generally lying between 1000 and 500 000, preferably between 10 000 and 250 000 and contain 25 to 70% of units derived from N-vinyl-pyrrolidone. The supports thus prepared can be utilized for the immobilization of target molecules in solution, in particular in procedures for the detection and estimation of nucleotide sequences. The layers of polymers adsorbed on such supports nonetheless have the disadvantage of being of very low stability, owing to the relatively small molecular size of these copolymers and the high percentage of lateral substituents (30 to 75%). Moreover, U.S. Pat. No. 6,692,914 describes sensors consisting of a substrate the surface whereof contains a plurality of segmented polymer chains (block polymers) in the form of a brush containing a water-soluble or water-dispersible segment and at least one probe selected from biological molecules, bound to said segment. The quality of such a sensor is closely linked to the density of the polymer brushes which is a parameter which is often difficult to control. Thus too low a density of the polymer brush will result in a surface displaying non-functionalized parts (holes) onto which the target molecules will be able to adsorb in non-specific ways while too high a density will result in a sensor on which only the functional groups located at the terminal end of the polymer chains will be accessible, which can also limit the sensitivity of the sensor.

There are moreover different cases, particularly in the context of proteomics, where molecules having a limited lifetime, for example enzymes, are attached to the surface of solid supports. By way of example, trypsin can be attached to the surface of reactors and, in that case, undergoes very little autolysis compared to trypsin in solution. With the aim of increasing the digestion rate of different proteins, it is in fact possible to maximize the surface (proteins attached to the surface) to volume (free proteins in solution) ratio. Usually, the attachment of the trypsin is effected on quartz or silica microspheres which are then inserted into microcapillaries which serve as trypsin reactors. In this case, the attachment of the trypsin is effected by means of a reduction reaction on a Schiff's base between a residual primary amine group of the trypsin and an aldehyde group borne by the surface (see in particular Muilin C., in “Methods in Enzymology”, Colowick S. P., Caplan N. O. Eds., Academic Press, New York, 1987, Vol 136). Such methods are however slow and laborious and are not entirely satisfactory. In particular, after the immobilization of the trypsin on the surface of the supports, parts of the surface remain which have not been modified, and which it is necessary to saturate in order to minimize non-specific adsorption reactions of molecules in solution (peptides and proteins for example).

It is therefore in order to remedy the totality of these disadvantages and to provide a solid support having at least one surface on which it is possible to immobilize chemical or biological molecules of interest and which is both simple to prepare and to regenerate while retaining good detection sensitivity and good stability over time that the inventors have perfected that which constitutes the object of the present invention.

The first object of the present invention is a water-soluble random (non-segmented) polymer containing probe molecules as lateral substituents, characterized in that:
it results from the copolymerization of an N,N-[dialkyl(C₁-C₄)acrylamide] monomer A and a monomer B selected from:
   a) monomers B1 containing at least one reactive chemical functional group capable of forming a covalent bond with a complementary chemical functional group of a probe molecule and
   b) probe molecules functionalized with a copolymerizable monomer (monomer B2),
      said probe molecules aforesaid in a) and b) being selected from proteins, and in particular proteins with enzymatic activity, antibodies, antigens and peptides; polysaccharides, oligosaccharides, and nucleic acids such as oligonucleotides, DNA and RNA; and small organic molecules, said lateral substituents being at least two in number per molecule of copolymer,
it displays a molecular weight which must be greater than 500 000 g/mole, and
the incorporation ratio of said probe molecules must be lower than 30% in number relative to the total number of N,N-[dialkyl(C₁-C₄)acrylamide] monomeric units.

The inventors have in fact discovered that the random water-soluble polymers as claimed in the present invention and as defined above are capable of being physically adsorbed in a stable manner on the surface of solid supports, and thus make it possible to obtain solid supports having at least one surface functionalized with probe molecules such as for example nucleic acid or protein chips on which it is then possible to immobilize target molecules of interest while minimizing the adsorption of other molecules in solution. Contrary to the polymers utilized to create the sensors described in U.S. Pat. No. 6,692,914, the polymers as claimed in the invention do not result in the formation of a brush of polymers adsorbed by one of their extremities at the surface of the support. Instead they form a layer of polymers adsorbed on the surface of the support within which [layer] the two terminal extremities of the polymer chain are free.

As claimed in the present invention, a small organic molecule is understood to mean organic molecules preferably displaying a molecular weight less than or equal to 1000 g/mole. Among such molecules, hydrocarbon molecules such as N-propyl methacrylate, N-decyl methacrylate and N-octadecyl acrylamide, and drugs and active principles, can in particular be mentioned.

As claimed in a preferred embodiment of the invention, said polymer has a molecular weight greater than or equal to 1×10⁶ g/mole. The inventors have in fact found that the stability of surfaces functionalized with such polymers was still greater when the polymers used had such a molecular weight.

Among the C₁-C₄ alkyl groups of the N,N-[dialkyl(C₁-C₄)acrylamide] monomers A which are constituents of the copolymers as claimed in the invention, the methyl, ethyl, n-propyl and n-butyl groups can be mentioned, the methyl group being quite particularly preferred. As the monomer A, N,N-dimethylacrylamide is thus quite particularly preferred.

Among the monomers B, vinylic and acrylamide monomers such as acrylic and methacrylic acids, N-aminoalkyl acrylamides, N-aminoalkyl methacrylamides, aminoalkyl acrylates and aminoalkyl methacrylates, wherein the alkyl group can for example represent the ethyl, propyl, butyl, pentyl or hexyl group, can in particular be mentioned.

Among the reactive chemical functional groups of the monomers B1, the carboxyl, amine, alcohol and thiol functional groups can be mentioned, which will be capable of interacting with a complementary functional group borne by said lateral substituents such as electron pair donors such as amines, alcohols, thiols, carboxyls and carbonyls. A more exhaustive list of complementary pairs of functional groups can easily be found in any monograph of organic chemistry.

As claimed in a preferred embodiment of the invention, the probe molecules present in the polymer as lateral substituents are preferably selected from active biological molecules displaying a limited lifetime, such as enzymes.

As claimed in another preferred embodiment of the invention, the incorporation ratio of the probe molecules lies between 1 and 20% by number relative to the total number of monomer units of N,N-[dialkyl(C₁-C₄)acrylamide] monomer A.

The polymers as claimed in the invention and as described previously can be prepared in accordance with the standard copolymerization techniques well known to the person skilled in the art such as for example copolymerization by the radical route, by reacting monomers A as defined above with monomers B1 or B2 as defined above in an appropriate solvent such as water, in the presence of a polymerization activator such as for example a redox couple such as the ammonium persulfate/sodium metabisulfite couple. The preparation of the monomers B2 can also be performed in accordance with standard methods well known to the person skilled in the art, for example by reacting, under anhydrous conditions in a suitable organic solvent such as dichloromethane, tetrahydrofuran (THF), dimethylformamide (DMF) or dimethyl sulfoxide (DMSO), a probe molecule containing a carboxyl group (biotin for example) with the vinyl group of a copolymerizable monomer such as for example N-aminopropyl methacrylamide, in the presence of a coupling agent such as for example dicyclohexylcarbodiimide; the reaction of a carboxyl group and an amine group being in fact well known for many years in the literature, in particular in the article by Khorana H. G. et al., Chem. Rev., 1953, 53, 145-166. Similarly, trypsin modified with a vinyl group can be prepared by the method described by Plate N. A. et al., Polymer Science USSR, 1989, 31, 216-219.

When the synthesis is completed, the polymers as claimed in the invention can be characterized by standard analytical techniques such as refractive index exclusion-diffusion chromatography and with detection by diffusion of light or by viscosimetry in order to gather information on the size of the polymers or else by nuclear magnetic resonance (NMR) techniques in order to gather information on their structure.

Also an object of the present invention is the utilization of at least one water-soluble random polymer as described above for the preparation of a solid support having at least one surface functionalized with probe molecules, and in particular for the preparation of protein or nucleic acid, in particular DNA, chips.

Also therefore an object thereof is a process for the preparation of a solid support having at least one surface functionalized with a water-soluble random polymer as claimed in the invention, characterized in that it comprises the following steps:
contacting of a solid support having at least one surface to be functionalized with a solution of at least one water-soluble random polymer as claimed in the invention and as defined above in a compatible solvent,
incubation of said surface with said solution of polymer for a time sufficient for the adsorption of the polymer onto the surface of the solid support,
rinsing of the solid support by means of a polymer-free solvent to obtain a solid support having at least one surface functionalized with an adsorbed layer of polymers.

The solid supports capable of being functionalized as claimed in this procedure are preferably selected from supports having at least one surface of the type of silica and derivatives thereof such as glass and quartz, or any other material covered with silica, glass or quartz.

As claimed in the invention, a compatible solvent is understood to mean any solvent enabling the dissolution of the polymer, not causing alteration in the probe molecules which it contains as lateral substituents and for which the surface of the solid support to be functionalized will have an affinity less than that which it will have for the polymers as claimed in the invention. Thus, on contacting of the solution of polymers with the support, the polymers in solution will be able to adsorb spontaneously onto the surface of the support. The selection of such a solvent depending on the nature of the polymers and of the surface to be functionalized can be effected by measuring the surface tension of the pure solvent (γs) and that of the polymers (γp) when these are liquid. When γs is greater than γp, the polymer will be capable of adsorbing onto the surface of the support when it is in solution in the solvent.

Such compatible solvents are generally selected from water and aqueous buffers such as for example ammonium bicarbonate or other saline buffers.

There are also two other determining factors with regard to the adsorption of the polymers onto the surface. The first factor is the level of interaction between the surface of the solid support and the constituent monomer units of the polymer, that is to say the quantity of energy necessary for the adsorption of the polymer (forces of attraction). The second factor is the reduction in the conformational states of the polymer on the surface of the solid support, that is to say the decrease in the entropy of the chain. This is due to the impenetrability of the surface for the monomer units of the polymer and corresponds to the energy which tends to repel the polymer from the surface of the solid support (repulsive forces). The thickness of the layer of polymers adsorbed onto the surface of the solid support will be greater the greater are the repulsive forces. The strength of the adsorption of the layer of polymers on the surface of the solid support will thus be a function of the ratio between the forces of attraction and the repulsive forces.

The duration of the incubation of the solid support with the polymers in solution preferably lies between 1 and about 60 minutes, and still more preferably between 5 and about 40 minutes.

As claimed in a preferred embodiment of this process, the quantity of polymers in solution lies between about 0.001% and 5% by weight relative to the volume of the solution of polymers and still more preferably between about 0.1% and 2% (weight/volume).

As claimed in a preferred embodiment of the process as claimed in the invention, the solvents used for rinsing the support after adsorption of the polymers and before their utilization are preferably selected from the solvents described above and utilized to make the solution of polymers. Still more preferably, the solvent utilized for rinsing the support is identical to that utilized for making the polymer solution.

Also an object of the present invention are the solid supports obtained by implementing the preparation process as claimed in the invention, said supports being characterized in that they have at least one surface functionalized with an adsorbed layer of water-soluble random polymers bearing probe molecules as lateral substituents as previously defined. Such supports can in particular take the form of a block, channel, capillary, reactor or reaction chamber such as for example the devices normally used for performing enzymatic reactions (enzymatic digestion).

As claimed in a preferred embodiment of the invention, the adsorption of the polymers onto the surface of the solid support is effected by means of the N,N-[dialkyl(C₁-C₄)acrylamide] monomers A.

These solid supports are in particular protein chips and in particular enzyme, peptide or polypeptide chips, nucleic acid and in particular DNA or RNA chips, oligosaccharide or polysaccharide chips. In such supports, the thickness of the polymer layer generally lies between 1 and 100 nm.

The thickness of the polymer layers can be measured for example by ellipsometry or by means of more elaborate techniques using evanescent waves (Allain C. et al., Phys. Rev. Lett., 1982, 49, 1694) or neutron diffusion (Barnett K. et al., “The effects of polymers on dispersion stability”, Tadros, J., Ed., Academic Press, 1982).

Also an object of the invention is the utilization of these supports for the immobilization and the screening of complementary target molecules in solution or the implementation of biochemical, chemical or biological reactions on solid support such as for example enzymatic reactions. As claimed in the invention, the word “biochemical” relates to reactions, processes and procedures which involve at least one substrate and its enzyme. By way of example, the words “biochemical reaction” can be utilized in the context of the present invention to refer to nucleic acid amplification processes such as polymerization chain reactions (PCR), the determination of a genotype such as microsequencing, or again the sequencing of nucleic acids. The word “biochemical” likewise includes other types of reaction catalyzed by an enzyme such as the digestion of proteins by proteases, the cleavage of DNA by nucleases, the phosphorylation of molecules by kinases, the isomerization of molecules by isomerases, the conversion of dopamine into norepinephrine by dopamine hydrolase, etc.

As claimed in the present invention, the word “chemical” is used to refer to reactions, methods and processes wherein there is at least one step involving a reaction which is not catalyzed by an enzyme. By way of example, the word “chemical” can be utilized to refer to syntheses of organic or inorganic molecules, degradation reactions one stage whereof is not catalyzed by an enzyme and also chemical reactions catalyzed by ultraviolet radiation.

Still as claimed in the present invention, the word “biological” is used to refer to reactions at least one step whereof involves a living organism such as a cell, a cell culture, a mass of adhering cells, a mono- or multicellular organism, parts of tissues or organs. The word “biological” utilized in the context of the present invention thus includes mono- or multicellular eukaryotic organisms, and also prokaryotic organisms such as bacteria and viruses.

Thus, also an object of the invention is a process for immobilization of target molecules and for screening of complementary target molecules in solution or for implementation of biochemical, chemical or biological reactions on solid support, characterized in that it comprises at least the following steps:
a) contacting a solid support having at least one surface functionalized with an adsorbed layer of water-soluble random polymers bearing probe molecules as lateral substituents with a liquid sample capable of containing target molecules, for a time sufficient for the effecting of said immobilization or said reaction,
b) desorption of the polymer layer from the surface of the support by rinsing of the support with an alkaline solution or a solvent,
c) if necessary, repetition of the above steps a) and b).

Through this procedure, it is in fact possible to perform a series of several immobilizations or of several reactions on the same support through the step of regeneration of the surface by means of the alkaline solution or the solvent which makes it possible to obtain a support whose surface can again be functionalized with a polymer bearing the same probe molecules or with a polymer bearing different probe molecules.

The alkaline solutions utilizable for performing the support regeneration step can be selected from solutions of sodium, potassium or ammonium hydroxide. The solvents utilizable for performing the support regeneration step are preferably selected from the solvents for which the adsorbed polymers have more affinity than for the surface onto which they were previously adsorbed.

Among such solvents, organic solvents which are miscible with water such as methanol, ethanol, isopropanol, acetonitrile, acetone, etc. can in particular be mentioned.

Preferably, step b) of the support regeneration is performed by means of a solution of sodium hydroxide.

Besides the above provisions, the invention also includes other provisions which will emerge from the description that is to follow, which relates to an example of preparation of a copolymer of N,N-dimethylacrylamide and acrylic acid containing trypsin as probe molecule, to an example of preparation of a glass capillary having a surface functionalized with such a polymer and to the utilization of such a capillary for performing a creatine digestion reaction, to an example of preparation of a copolymer of N,N-dimethylacrylamide and biotin functionalized with a vinyl group, to an example of preparation of a solid support having a surface modified with such a copolymer and its utilization for the immobilization of target biotinylated molecules by means of streptavidin.

It must however be clearly understood that these examples are given solely by way of illustration of the object of the invention, whereof they in no way constitute a limitation.

### EXAMPLE 1

### Preparation of a Copolymer of N,N-Dimethylacrylamide and Acrylic Acid P(DMA-s-AA) Bearing Molecules of Trypsin as a Probe Molecule

1) Synthesis of the Copolymer P(DMA-s-AA)

2.7 g of N,N-dimethylacrylamide (0.0272 mol) and 0.3 g of acrylic acid (0.0042 mol) in 30 ml of MilliQ water are introduced into a three-necked flask. The mixture is brought to basic pH (between 8 and 10) by addition of 3N caustic soda in order to avoid any side reaction which might disturb the control of the molecular weights. The mixture is then stirred for 20 minutes with vigorous nitrogen sparging to eliminate dissolved oxygen.

The reaction mixture is then brought up to a temperature of 32° C. by means of a water-bath. 1 mol % of ammonium peroxydisulfate and 0.1 mol % of sodium metabisulfite relative to the quantity of monomers are added in order to initiate the copolymerization reaction. The reaction is carried out for 1 hour and 30 minutes, the mixture becoming very viscous after about 45 minutes. The reaction medium is then diluted to 500 ml with MilliQ water, acidified to a pH of 3 with 3N hydrochloric acid, then ultrafiltered on 100 000 g/mole membranes and finally lyophilized.

The reaction product was characterized by proton NMR and acid-base assay in order to determine the acrylic acid incorporation ratio. A P(DMA-s-AA) copolymer is obtained wherein, for an initial proportion of 10% by weight of acrylic acid, 4.8% (by weight) are incorporated in the copolymer.

2) Grafting of the Trypsin

In this step, the grafting of the trypsin onto the P(DMA-s-AA) copolymer prepared above in Step 1 is effected via its N-terminal extremity which reacts with the carboxyl groups of the acrylic acid to form an amide. The grafting procedure utilized is based on the EDC/NHS (1-ethyl-3-(3-dimethylaminopropyl)carbodiimide hydrochloride/N-hydroxysuccinimide) couple.

To do this, 100 mg of P(DMA-s-AA) prepared above in Step 1 are dissolved in 10 ml of 0.1 M 2-(N-morpholino)ethanesulfonic acid (MES) containing 0.5 M of sodium chloride and having a pH of 6. When the copolymer has dissolved, 52 mg (2.71×10⁻⁴ mol) of EDC and 63 mg (5.47×10⁻⁴ mol) of NHS are added, each of these two compounds having previously been dissolved in 1 ml of 0.1 M MES containing 0.5 M of NaCl and having a pH of 6.

The reaction medium is stirred at ambient temperature for 15 minutes then filtered on a Microcon® membrane having a cutoff threshold of 100 000 g/mole so as to remove the unreacted NHS and EDC. The reaction mixture is then centrifuged at 10 000 rpm for 1 hour and 15 minutes.

The reaction mixture is then brought to a temperature of 4° C. then 2.4 mg (1.04×10⁻⁷ mol) of a 2 mg/ml solution of trypsin in 0.1 M MES, 0.5 M NaCl, pH 6 are added. The mixture is then maintained at a temperature of 4° C. with stirring for 12 hours.

At the end of the reaction, the reaction mixture is diluted to 300 ml by addition of MilliQ water then ultra-filtered on membranes having a cutoff threshold of 100 000 Da in order to remove unreacted trypsin and the secondary reaction product (NHS) and to desalt the medium. The reaction medium is then concentrated to a volume of 50 to 100 ml then lyophilized.

### EXAMPLE 2

### Preparation of a Glass Capillary Functionalized with a Copolymer Bearing Trypsin as Probe Molecule

The trypsinated copolymer as prepared above in Example 1 is suspended in 25 mM NH₄HCO₃ buffer (pH 8), filtered on a 0.22 μm filter, in a proportion of 3% (w/v) of trypsinated copolymer/ml of buffer.

A capillary of 20 cm length and 75 μm diameter (Polymicro®) is treated by passing the following solutions through it: 3 N NaOH, 25 mM NH₄HCO₃ buffer, 0.2 M HCl, buffer and finally the solution of trypsinated P(DMA-s-AA). This last solution is allowed to incubate with the capillary for 30 minutes then a final NH₄HCO₃ rinsing is performed.

A glass capillary is thus obtained whose internal surface is functionalized with an adsorbed layer of trypsinated P(DMA-s-AA) water-soluble random polymer.

This type of capillary can then be utilized for the digestion of creatinine by the standard techniques well known to the person skilled in the art, for example by passing a solution of creatinine in NH₄HCO₃ through it, and analyzing the digestion product by mass spectrometry.

### EXAMPLE 3

### Preparation of a Copolymer of N,N-Dimethylacrylamide and Biotin Functionalized with a Vinyl Group

This example illustrates the synthesis of a copolymer of N,N-dimethylacrylamide and biotin functionalized with a vinyl group. The functionalization of the biotin is effected by reaction of a carboxyl group of the biotin with N-aminopropyl methacrylamide containing a vinyl group. The reaction is performed under anhydrous conditions in an organic solvent such as dichloromethane, tetrahydrofuran (THF), dimethylformamide (DMF) or dimethyl sulfoxide (DMSO) by means of a coupling agent such as dicyclohexyl carbodiimide.

The copolymerization of the biotin with the N,N-dimethylacrylamide monomer is performed under the same conditions as those described above in Example 1 by reacting 2.7 g of N,N-dimethylacrylamide (0.0272 mol) and 1.2 g (0.0042 mol) of biotin modified with a vinyl group.

A copolymer is obtained which is purified by ultra-filtration by means of a filter with a cutoff threshold of 100 000 Da then lyophilized.

### EXAMPLE 4

### Preparation of a Solid Support Having a Surface Modified with a Copolymer of N,N-Dimethylacrylamide and Biotin and its Utilization for the Immobilization of Biotinylated Target Molecules by Means of Streptavidin

The purified copolymer as prepared above in Example 3 is dissolved in a buffer at the desired concentration, that is to say generally between 0.1 and 1% by weight, then adsorbed on the surface of a capillary. Avidin or streptavidin can then be coupled to the copolymer attached to the surface of the capillary. The resulting copolymer-avidin or copolymer-streptavidin complex can then be utilized for capturing biotinylated molecules in solution such as for example proteins, peptides, oligonucleotides, antibodies, etc.

1. A water-soluble random polymer containing probe molecules as lateral substituents, characterized in that:
it results from the copolymerization of an N,N-[dialkyl(C₁-C₄)acrylamide] monomer A and a monomer B selected from probe molecules functionalized with a copolymerizable monomer,
said probe molecules aforesaid in a) and b) being selected from proteins;
polysaccharides, oligo-saccharides, nucleic acids and small organic molecules, said lateral substituents being at least two in number per molecule of copolymer,
it displays a molecular weight which must be greater than 500 000 g/mole, and
the incorporation ratio of said probe molecules must be lower than 30% in number relative to the total number of N,N-[dialkyl(C₁-C₄)acrylamide] monomeric units.

2. The polymer as claimed in claim 1, characterized in that the proteins are selected from enzymes, antibodies, antigens and peptides and the nucleic acids are selected from oligonucleotides, DNA and RNA.

3. The polymer as claimed in claim 1, characterized in that the small organic molecules are organic molecules displaying a molecular weight less than or equal to 1000 g/mole.

4. The polymer as claimed in claim 1, characterized in that it has a molecular weight greater than or equal to 1×10⁶ g/mole.

5. The polymer as claimed in claim 1, characterized in that the C₁-C₄ alkyl groups of the N,N-[dialkyl(C₁-C₄)acrylamide] monomers A are selected from the methyl, ethyl, n-propyl and n-butyl groups.

6. The polymer as claimed in claim 5, characterized in that the N,N-[dialkyl(C₁-C₄)acrylamide] monomer A is an N,N-dimethylacrylamide monomer.

7. The polymer as claimed in claim 1, characterized in that the monomers B are selected from vinylic and acrylamide monomers.

8. The polymer as claimed in claim 1, characterized in that the probe molecules are selected from enzymes.

9. The polymer as claimed in claim 1, characterized in that the incorporation ratio of the probe molecules lies between 1 and 20% by number relative to the total number of monomer units of N,N-[dialkyl(C₁-C₄)acrylamide] monomer A.

10. The utilization of at least one water-soluble random polymer as defined in claim 1 for the preparation of a solid support having at least one surface functionalized with probe molecules.

11. The utilization as claimed in claim 10, characterized in that the solid support is a protein or nucleic acid chip.

12. A process for the preparation of a solid support having at least one surface functionalized with a layer of water-soluble random polymers, characterized in that it comprises the following steps:
contacting of a solid support having at least one surface to be functionalized with a solution of at least one polymer as defined above in claim 1 in a compatible solvent,
incubation of said surface with said solution of polymer for a time sufficient for the adsorption of the polymer onto the surface of the solid support,
rinsing of the solid support by means of a polymer-free solvent to obtain a solid support having at least one surface functionalized with an adsorbed layer of polymers.

13. The process as claimed in claim 12, characterized in that the solid supports are selected from supports having at least one surface of the type of silica and derivatives thereof.

14. The process as claimed in claim 13, characterized in that the silica derivatives are glass and quartz.

15. The process as claimed in claim 12, characterized in that the duration of the incubation of the solid support with the polymers in solution lies between 1 and 60 minutes.

16. The process as claimed in claim 12, characterized in that the quantity of polymers in solution lies between 0.001% and 5% by weight relative to the volume of the solution of polymers.

17. A solid support obtained by implementing the preparation process as defined in claim 12, characterized in that it has at least one surface functionalized with an adsorbed layer of water-soluble random polymers bearing probe molecules as lateral substituents.

18. The solid support as claimed in claim 17, characterized in that it takes the form of a contact, channel, capillary, reactor or reaction chamber.

19. The solid support as claimed in claim 17, characterized in that the thickness of the polymer layer lies between 1 and 100 nm.

20. The utilization of a solid support as defined in claim 17 for the immobilization and the screening of complementary target molecules in solution or the implementation of biochemical, chemical or biological reactions on solid support.

21. A process for immobilization of target molecules and for screening of complementary target molecules in solution or for implementation of biochemical, chemical or biological reactions on solid support, characterized in that it comprises at least the following steps:
a) contacting a solid support having at least one surface functionalized with an adsorbed layer of water-soluble random polymers bearing probe molecules as lateral substituents as defined in claim 17 with a liquid sample capable of containing target molecules, for a time sufficient for the effecting of said immobilization or said reaction,
b) desorption of the polymer layer from the surface of the support by rinsing of the support with an alkaline solution or a solvent, and
c) if necessary, repetition of the above steps a) and b).

22. The process as claimed in claim 21, characterized in that the alkaline solution used for performing the support regeneration step is selected from sodium, potassium or ammonium hydroxide.

23. The process as claimed in claim 21, characterized in that the solvent used for performing the support regeneration step is selected from organic solvents which are miscible with water.
